(19) United States
(12) Patent Application Publication
(10) Pub. No.: US 2007/0274962 A1
(43) Pub. Date: Nov. 29, 2007

(54) Methods and Composition for Transplantation of Dopaminergic Neurons for Parkinson's Disease

(76) Inventor: Ge Ming Lui, Honolulu, HI (US)

Correspondence Address: JACOBSON HOLMAN PLLC, 400 SEVENTH STREET N.W., SUITE 600, WASHINGTON, DC, 20004, US

(21) Appl. No.: 10/580,547

(22) PCT Filed: Dec. 2, 2004
(86) PCT No.: PCT/US04/40178

§ 371 Date: Apr. 13, 2007

Related U.S. Application Data
(60) Provisional application No. 60/526,684, filed on Dec. 2, 2003.

(51) Int. Cl. A61K 35/12; A61P 25/16

(52) U.S. Cl. 424/93.7

(57) ABSTRACT

This invention discloses methods to attach and grow a monolayer of cultured human retinal pigment epithelial cells (RPE) for use in implantation into the brain as a treatment for Parkinson's disease. The invention will enable the delivery of the transplanted RPE cells in microcarriers composed of intergratable or degradable substrates, including glass, plastic, polymer gels, gelatin and collagen, and glycosaminoglycans (GAGS). The invention involves the coating of the microcarrier surface with diamond-like carbon, alone or in combination with attachment factors such as laminin, fibronectin, RGDS, and extracellular matrix to increase the attachment of the RPE to the surface. Additionally the invention discloses the use of bFGF conjugated with polycarbophil, EGF conjugated with polycarbophil and heparin sulfate as also being incorporated into the micro carrier to augment attachment and proliferation of RPE during transplantation.

This patent application claims priority to U.S. patent application Ser. No. 60/526,684 filed Dec. 2, 2003, and is a continuation-in-part of PCT Application No.: PCT/US04/33194, and are both incorporated by reference herein as if set forth in its entirety.

### BACKGROUND OF THE INVENTION

1. Field of Invention

This patent application describes the culturing and implantation of cultured human retinal pigment epithelial (RPE) cells for the transplantation of these cells into the brain for treatment of Parkinson's Disease.

2. Description of Prior Art

Parkinson's disease is a progressive neurological disorder which affects the aging population. It is manipulated by a cluster of motor and cognitive dysfunction, muscle tremor, bradykinesis, and rigidity. The causes of these symptoms are attributed to the decrease in production of the neurotransmitter dopamine (DA) by the DA producing cells in the substantia nigra, thus resulting in the drop of DA level to less than 60% of the normal levels in the striatum (RL Watts, et al., (2003) J. Neural Transm. [supp} 65:215-227).

Cell therapy of various types has been attempted, including stereotactic striatal implantation of DA-producing human fetal mesencephalic neural tissues. Such procedures are known in the are and are described in (Fahn, S. (2000 Mov. Disord. 15 [Suppl. 3]# M114; Freed C R, et al., (2001) N. Engl. J. Med. 344: 710-719; Dunnett S B, and Bjorklund A (1999) Nature 399: S32-S39) and are incorporated herein by reference. Several studies in a rat model have suggested embryonic stem cell implantation may generate differentiation of the stem cells into dopaminergic neurons (Deacon T et al., (1998) Exp. Neurol. 149: 28-41; Englund U et al., (2002) Proc. Natl. Acad. Sci. USA 99: 17089-17094). Human RPE cells, however, are a readily available cell type under tissue culture conditions. These cells are natural producers of L-DOPA, a precursor of DA as well as dopamine (Cherksey B D (1994) Exp. Neurol. 129:S18; Marchionini D M, et al. (1999) Abstracts Am. Soc. Neural. Transpl. Repair 5: A-05). It is therefore the best candidate for cell transplantation in the treatment of Parkinson's disease.

However, a problem stands in between the insertion of the cells into the substantia nigra and the survival of the transplanted cells. Cell suspensions that are not protected when introduced into the brain stem, have demonstrated a very low survival rate. This is attributed to the cell loss during the injection process and the destruction of unattached cells by the host immune system. More recently, a microcarrier system has been used for carrying the cells into the brain stem to improve the survival rate. See U.S. Pat. Nos. 5,618,531, 5,750,103 and 6,060,048, all of which are incorporated by reference herein.

Cell transplantation has been proposed as an alternative for total organ replacement for a variety of therapeutic needs, including treatment of diseases in the eye, brain, liver, skin, cartilage, and blood vessels. See, for example, J P Vacanti et al., J. Pediat. Surg., Vol. 23, 1988, pp. 3-9; P Aebischer et al., Brain Res. Vol. 488, 1998, pp. 364-368; C B Weinberg and E. Bell, Science, Vol. 231, 1986 pp. 397-400; I V Yannas, Collagen III, M E Nimni, ed., CRC Press, Boca Raton, 1988; G L Bumgardner et al., Hepatology, Vol. 8, 1988, pp. 1158-1161; A M Sun et al., Appl. Bioch. Biotech., Vol. 10, 1984, pp. 87-99; A A Demetriou et al., Proc. Nat. Acad. Sci. USA, Vol. 83, 1986, pp. 7475-7479; W T Green Jr., Clin. Orth. Rel. Res., Vol 124. 1977, pp. 237-250; C A Vacanti et al., J. Plas. Reconstr. Surg., 1991; 88:753-9; P A Lucas et al., J. Biomed. Mat. Res., Vol. 24, 1990, pp. 901-911. The ability to create human cell lines in tissue culture will enhance the prospect of cell transplantation as a therapeutic mode to restore lost tissue function. It is especially vital to be able to create human cultured cell lines from tissues of the neural crest, since tissues or organs derived from that origin couldn't usually repair itself from damage after an individual reaches adulthood.

Conventional tissue culture lab wares useful in growing cells in vitro, are usually coated with a negative charge to enhance the attachment and sometimes proliferation of mammalian cells in culture. However, traditionally it has been most difficult to achieve a satisfactory attachment, maintenance, and propagation of mammalian neuronal cells with the conventional tissue culture surfaces. Improvements have been made by adding layers of collagen gel or depositing an extracellular matrix secreted by rat EHS tumor cells onto the tissue culture plates and dishes to facilitate neural cell attachment and proliferation. These techniques, however, are hindered by the shortcoming that the material has to be layered on the culture surfaces shortly before the cells are seeded.

The use of a biopolymer carrier to support the attachment, growth, and eventually as a vehicle to carrying the cells during transplantation is vital to the success of cell replacement therapy, particularly in the brain and the back of the eye, where cells derived from the neural crest origin is often damaged during the aging process. There are seven general classes of biopolymers: polynucleotides, polyamides, polysaccharides, polyisoprenes, lignin, polyphosphate and polyhydroxyalkanoates. See for example, U.S. Pat. No. 6,495,152. Biopolymers range from collagen IV to polyorganosiloxane compositions in which the surface is embedded with carbon particles, or is treated with a primary amine and optional peptide, or is co-cured with a primary amine-or carboxyl-containing silane or siloxane, (U.S. Pat. No. 4,822,741), or for example, other modified collagens are known (U.S. Pat. No. 6,676,969) that comprise natural cartilage material which has been subjected to defatting and other treatment, leaving the collagen II material together with glycosaminoglycans, or alternatively fibers of purified collagen II may be mixed with glycosaminoglycans and any other required additives. Such additional additives may, for example, include chondronectin or anchorin II to assist attachment of the chrondocytes to the collagen II fibers and growth factors such as cartilage inducing factor (CIF), insulin-like growth factor (IGF) and transforming growth factor (TGFβ).

The current method to avoid cell death is to attach the pigmented cells onto glass beads and then injecting the cells-bead combination into the brain. While keeping the cells viable, the glass bead is non-immunogenic and therefore causes no immune reaction. However, there is no way to retrieve the glass beads once they were injected and in the long run, this glass beads may become a cause of concern since some may break and cause injury. Our approach is to use a biodegradable polymer-gel to encapsulate the pigmented cells immediately after they are injected into the brain via light activation. The transplanted cells will therefore be protected and be able to perform their function, the polymer will degrade with time while the transplanted cells incorporated themselves into the system.

Until the advent of the present invention in conjunction with the methods outlined in PCT/US04/333194, it was not possible to culture mammalian or human neuronal tissues from the neural crest or individual neurons and get them to grow and divide in vitro.

In transplanting cultured RPE cells into the brain via injection, it was observed in animal model that the cells either died or lost their function if they were injected without attaching to a support (i.e. when injected as a cell suspension). Consequently when they were attached onto small glass beads and then injected into the brain, they retained their functions and relieved the Parkinson's symptoms. The method we describe to encapsulate the pigmented cells with light sensitive polymer-gel will serve to prevent the cells from dying or losing functions after their introduction into the brain.

### SUMMARY OF THE INVENTION

One aspect of the present invention is the disclosure of methods of coating tissue culture lab ware with a stable layer of carbon plasma, most preferably the DLC that can enhance the attachment and growth of neuronal cells, and can provide a ready supply of apparatus for successful the tissue culture of these cell types.

It is an object of the present invention to create a specialized attachment and survival platform for the adhesion of RPE cells or other neuronal cells on the microcarriers during the cell transplantation process. The DLC coating can be deposited onto microcarriers that are composed of glass, plastics, biopolymer gels, collagen and gelatin, GAGS, synthetic polymers, and metal. The DLC coat can be added on top of other types of coatings such as extracellular matrix (ECM), adhesive molecules, and growth factors.

Human or mammalian cells from the neural crest origin or neurons in particular, are known to exhibit two difficult behaviors. One is that they do not usually replicate in vivo or under tissue culture conditions, and secondly they do not attach very well to conventional cell culture surfaces. By coating a surface with carbon plasma, known as diamond-like carbon (DLC), the inventors have found that neurons will readily attach to the culture surface and exhibit a proliferation response.

The mechanical and tribological properties of DLC films (friction coefficient around 0.1 in air, hardness up to about 80 GPa, and elastic modulus approaching 600 GPa) are very close to those of diamond. Moreover, these films are chemically inert in most aggressive environments, and may be deposited with densities approaching that of diamond. However, in contrast to carbon vapor deposition, diamond, DLC films are routinely produced at room temperature, which makes them particularly attractive for applications where the substrate cannot experience elevated temperatures.

It is another object of the present invention to teach the deposition of a DLC coat onto a biopolymer surface, which in turn will support the attachment and growth of human and mammalian neurons, as well as other cell types originating from the neural crest.

A further object of the present invention is to create a specialized tissue culture platforms for the growth and maintenance of neuronal cells and cells of neural crest origin in vitro for the purpose of propagation of cell lines and performing experiments. The DLC coated products of the present invention include tissue culture dishes, flasks, slides, filter chambers, polymer and glass beads, sheets, and blocks. The coating can be deposited onto plastic, glass, synthetic and natural biopolymers, and metal. The DLC coat can be added on top of other types of coating such as extracellular matrix (ECM) secreted by cultured bovine corneal endothelial cells, adhesive molecule coating and growth factor coating to generate an improved product for specific human and mammalian cell growth.

In addition, the biopolymer used in the present invention, can be of natural or synthetic in origin. Natural biopolymers comprise collagen and other well known polymeric substances. For synthetic polymers, they can be acrylic and derivatives or copolymers such as polymethyl methacrylate, poly-N-isopropylacrylamide or poly-2-hydroxymethacrylate, polyvinyl alcohols and derivatives and copolymers. The biopolymer can either be a thin sheet or in microparticle form. To improve the growth supporting properties of the biopolymer, attachment or growth promoting factors can be embedded or incorporated into its composition during synthesis. Furthermore, a three dimensional growth medium suitable for supporting the growth and replication of neural cells comprising of a semi-solid biopolymer can also be coated with DLC to enhance its capability to support neuronal growth and maintenance. The biopolymer can also be comprised of chitosan or sodium alginate “may polymer” as well.

It is yet another object of the present invention to provide for the deposition of DLC onto the surface of interest via use of a plasma gun in a vacuum environment. Use of such a system is very flexible and therefore can be utilized to coat surfaces of many shapes and types.

These and other objects of the invention, as well as many of the attendant advantages thereof, will become more readily apparent when reference is made to the following detailed description of the preferred embodiments.

### DETAILED DESCRIPTION AND PREFERRED EMBODIMENTS

In describing a preferred embodiment, of the invention specific terminology will be resorted to for the sake of clarity. However, the invention is not intended to be limited to the specific terms so selected, and it is to be understood that each specific term includes all technical equivalents which operate in a similar manner to accomplish a similar purpose.

The methods described in the present invention will allow the coating of a polymer surface with DLC and similar coatings to render it useful as a carrier for cells derived from neural crest origin. The biopolymer can be a biodegradable moiety. The biopolymer can either be in the form of a thin sheet, in microparticle form, or as a semi-solid block. The biopolymer is coated with by using a plasma gun which will deposit a thin layer of carbon plasma with the thickness of 200 to 400 Å on to the intended culture surface.

Similar to diamond-like carbon (DLC) coating, amorphous carbon nitride (C—N) films can be extremely hard and wear-resistant. They may serve as candidates for the solution to the problem of aseptic loosening of total hip replacements. It has been reported by Du et al., that morphological behavior of osteoblasts on silicon, DLC-coated silicon and amorphous C—N film-deposited silicon in organ culture was investigated by scanning electron microscopy. Cells on the silicon wafers were able to attach, but were unable to follow this attachment with spreading. In contrast, the cells attached, spread and proliferated on the DLC coatings and amorphous C—N films without apparent impairment of cell physiology. The morphological development of cells on the coatings and films was similar to that of cells in the control. The results support the biocompatibility of DLC coating and are encouraging for the potential biomedical applications of amorphous C—N films in the present invention (C. Du et al., Biomaterials. April-May 1998;19(7-9):651-8.

The DLC Coating Process is as Follows:

The plasma equipment consists of a vacuum arc plasma gun manufactured by Lawrence Berkeley National Laboratory, Berkeley, Calif., that is operated in repetitively-pulsed mode so as to minimize high electrical power and thermal load concerns. The fitted with a carbon cathode, the plasma gun forms a dense plume of pure carbon plasma with a directed streaming energy of about 20 eV. The plasma is injected into a 90° magnetic filter (bent solenoid) so as to remove any particulate material from the cathode, and then transported through a large permanent magnet multipore configuration that serves to flatten the radial plasma profile; in this way the carbon plasma deposition is caused to be spatially homogenous over a large deposition area.

To yet further enhance the film uniformity, the substrate(s) to be DLC coated are positioned on a slowly rotating disk, thus removing and azimuthal inhomogeneity. The apparatus described was used to form DLC films of about 2 to 4000 Å thick, preferably about 200-400 Å thick.

To improve the ability of the biopolymer in supporting cell growth or attachment, an attachment mixture comprising of one or more of the following will be embedded or incorporated into its composition during synthesis: fibronectin at concentrations ranging from 1 μg to 500 μg/ml of polymer gel, laminin at concentrations ranging from 1 μg to 500 μg/ml of polymer gel, RGDS at concentrations ranging from 0.1 μg to 100 μg/ml of polymer gel, bFGF conjugated with polycarbophil at concentrations ranging from 1 ng to 500 ng/ml of polymer gel, EGF conjugated with polycarbophil in concentrations ranging from 10 ng to 1000 ng/ml of polymer gel, NGF at concentrations of ranging from 1 ng to 1000 ng/ml of the polymer gel and heparin sulfate at concentrations ranging from 1 μg to 500 μg/ml of polymer gel.

In the thin sheet or microparticle forms, the coated biopolymer, in a preferred embodiment, is used as a carrier for neural cell growth and as a vehicle for cell delivery during a cell transplantation procedure. The semi-solid polymer block form can be used as a neural cell maintenance device in coupling with an integrated circuit chip or a CCD chip to function as a neural stimulation detector. The coated surface can be further improved by coating with an extracellular matrix deposited by cultured bovine corneal endothelial cells and then subsequently overlaid with a DLC coating.

### EXAMPLE 1

### Coating a Biopolymer in the Form of a Sheet with DLC

The biopolymer sheets can be any dimension, preferably about 2 cm×2 cm of the present invention are fixed to a rotating disk which is in turn set up in the DLC coating chamber on top of a slowly rotating motor. The plasma equipment will generate a dense plume of pure carbon plasma via an ejecting gun with a directed streaming energy of about 20 eV. The plasma is injected into a 90° magnetic filter to remove any particulate material to form a high quality, hydrogen free diamond-like carbon. When transported through a large permanent magnet multipore configuration that serves to flatten the radial plasma profile, a carbon plasma deposition will be spatially homogenous over a large deposition area. As the carbon plasma plume approaches the slowly rotating disk holding the polymer sheet, a uniform film of DLC will coat the surface of the sheet. The sheet can be used for growing many kinds of cells, and preferably neuronal cells, or as a vehicle for cell transplantation after sterilizing with UV radiation or 70% alcohol rinse.

### EXAMPLE 2

### Coating of Biopolymer in the form of Microparticles with DLC

The biopolymer microparticles will be placed into a specialized rotating chamber and a plume of carbon plasma is generated as previously described in Example 1. The plasma gun will introduce the spray of DLC into the chamber while it is rotated slowly in a vertical axis. The microcarrier beads will be induced to suspend by an air current in the coating chamber, the beads are allowed to rise and descend in the alternating air current many times while the plasma gun is in operation to insure uniform coating of all sides. This process will be sustained over a period of about 2-3 hours to insure uniform and complete covering of all particle surfaces. A thin layer of DLC at the uniform thickness of about 200-400 Å will be deposited on the entire spherical surface. The product can then be sterilized by UV irradiation or alcohol rinse, packaged and sealed, and stored on the shelf until used.

### EXAMPLE 3

### Biopolymers with Attachment or Growth Promoting Factors Embedded or Incorporated into its Composition During Synthesis and Subsequently coated with DLC

The biopolymer of the present invention can be embedded with, or incorporated into its composition during synthesis, attachment or growth promoting factors comprising of one or more of the following: fibronectin at concentrations ranging from 1 μg to 500 μg/ml of polymer gel, laminin at concentrations ranging from 1 μg to 500 μg/ml of polymer gel, RGDS at concentrations ranging from 0.1 μg to 100 μg/ml of polymer gel, bFGF conjugated with polycarbophil at concentrations ranging from 1 ng to 500 ng/ml of polymer gel, EGF conjugated with polycarbophil in concentrations ranging from 10 ng to 1000 ng/ml of polymer gel, NGF at concentrations of ranging from 1 ng to 1000 ng/ml of the polymer gel and heparin sulfate at concentrations ranging from 1 μg to 500 μg/ml of polymer gel. The biopolymer is then made into thin sheet or a semi-solid bloc, and DLC deposition can be achieved as previously described in Example 1. Or the polymer can be made into micro-particles or spheres, and DLC deposition can be achieved as previously described in Example 2.

### EXAMPLE 4

### Coating of Biopolymer with Extracellular Matrix Deposited by Cultured Bovine Corneal Endothelial Cells and Subsequent Coating of the Sheet or Microparticles with DLC

The biopolymer sheet, and block of microparticles can first be coated with an extracellular matrix (ECM) prior to the DLC deposition on the culture surface. To achieve this, bovine corneal endothelial cells (BCE) are seeded at low density (about 2000 to 150,000 cells/ml, preferably about 20,000 cells/ml) onto the surface of the sheet or block, or allowed to attach to the surface of the microparticles. The BCE cells are maintained in culture medium containing DME-H16 supplemented with 10% calf serum, 5% fetal calf serum, 2% Dextran (40,000 MV) and 50 ng/ml of bFGF. The cells are incubated at 37° C. in 10% CO₂ for 7 days, during which time bFGF at a concentration of 50 ng/ml is added every other day. The BCE cells are removed by treating the polymer sheet, block, or microparticles with 20 mM ammonium hydroxide for 5 minutes. Then the biopolymer with the extracellular matrix coat is washed ten times with sufficient volume of PBS. After drying, the ECM coated polymer sheet or block is subjected to DLC deposition as previously described in Example 1, whereas the ECM-coated microparticles is subjected to DLC deposition as described in Example 2. After the sequential coating with ECM and DLC, the polymer sheet, block, or microparticle will be sterilized by UV irradiation or alcohol rinse, and used for neural cell growth or as a vehicle for cell transplantation.

### EXAMPLE 5

### A Substrate Containing a Biopolymer having Neurons Electrically Connected to an Integrated Circuit

The biopolymer of the present invention can be embedded with, or incorporated into its composition during synthesis, attachment or growth promoting factors comprising of one or more of the following: fibronectin at concentrations ranging from 1 μg to 500 μg/ml of polymer gel, laminin at concentrations ranging from 1 μg to 500 μg/ml of polymer gel, RGDS at concentrations ranging from 0.1 μg to 100 μg/ml of polymer gel, bFGF conjugated with polycarbophil at concentrations ranging from 1 ng to 500 ng/ml of polymer gel, EGF conjugated with polycarbophil in concentrations ranging from 10 ng to 1000 ng/ml of polymer gel, NGF at concentrations of ranging from 1 ng to 1000 ng/ml of the polymer gel and heparin sulfate at concentrations ranging from 1 μg to 500 μg/ml of polymer gel. The biopolymer is then made into thin sheet or a semi-solid bloc, and DLC deposition can be achieved as previously described in Example 1. Or the polymer can be made into micro-particles or spheres, and DLC deposition can be achieved as previously described in Example 2.

On the DLC coated substrate, an integrated circuit or chip has been set in place. As described in Zeck, G. & Fromherz, Proc. Nat. Acad. Sci., 98, 10457-10462, (2001), nerve cells will be placed on a silicon chip with a DLC coating, and then the nerve cells are fenced in place with microscopic plastic pegs. Neighboring cells will grow connections with each other and with the chip. A stimulator beneath each nerve cell will create a change in voltage that will trigger an electrical impulse to travel through the cell. Electrical pulses applied to the chip will pass from one nerve cell to another, and back to the chip to trip a silicon switch.

### EXAMPLE 6

### DLC Deposition on the Culture Surface of Tissue Culture Lab Ware

In the event of a flat culture surface such as a dish, filter insert, chamber slide, sheets, and blocks, the wares can be presented to the plasma gun with the culture surface upwards in the vacuum chamber, and the coating process can proceed as previously described. In the case of the microcarrier beads, they need to be induced to flow in the chamber to insure uniform coating on all sides. For enclosed surfaces like flasks and tubes, a special modified plasma gun will be inserted into the vessel and coat the desired surface. A thin layer of DLC at the uniform thickness of about 20 to about 4000 Å, preferably about 200-400 Å will be deposited onto the culture surface. The products can then be sterilized by UV irradiation or alcohol rinsing, packaged, sealed, and stored on the shelf until use.

### EXAMPLE 7

### Sequentially Coating the Culture Surface with ECM Secreted by Cultured Bovine Corneal Endothelial Cells and then DLC Deposition

In this embodiment, sparse cultures (about 1000 to about 50,000 cells/ml, preferably 2000-5000 cells/ml) of bovine corneal endothelial cells are seeded onto the culture surface of the intended lab ware, which includes dishes, flasks, tubes, filter inserts, chamber slides, microcarrier beads, roller bottles, cell harvesters, sheets, and blocks. The cells are maintained in a medium containing DME-H16 supplemented with 10% calf serum, 5% fetal calf serum, 2% Dextran (40,000 MV), and bFGF at Song/ml. The bovine corneal endothelial cells are grown for 7-10 days until confluence with bFGF added every other day at 50 ng/ml. Then the culture medium is removed and the cells are treated with sufficient 20 mM ammonium hydroxide in distilled water for 3 to 30 minutes. The surface is then washed with a sufficient amount of PBS 10 times to remove and residual ammonium hydroxide and dried in a sterile laminar flow hood. The coating of DLC can then be performed as previously described on top of the extracellular matrix. The product is then sterilized under UV radiation or alcohol rinse, and will be packaged, sealed, and stored on the shelf until use.

### EXAMPLE 8

### Sequential Coating of the Culture Surface by Attachment or Growth Promoting Reagents Followed by DLC Deposit

In this alternate embodiment, one or more of the attachment or growth promoting reagents comprised of fibronectin at concentrations ranging from 1 μg to 500 μg/ml, laminin at concentrations ranging from 1 μg to 500 μg/ml, RGDS at concentrations ranging from 0.1 μg to 100 μg/ml, bFGF conjugated with polycarbophil at concentrations ranging from 1 ng to 400 ng/ml, EGF conjugated with polycarbophil in concentrations ranging from 10 ng to 1000 ng/ml. The attachment or growth promoting reagents will be added to the culture surface, and then will be incubated at 4° C. for 20 minutes to 2 hours. The surface is then rinsed with PBS three times and dried in a sterile laminar flow hood. Then the product will be deposited with a DLC layer on top of the attachment or growth promoting reagent coat on the culture surface. The lab ware will then be sterilized by UV irradiation or alcohol rinse, packaged, sealed, and stored until use.

### EXAMPLE 9

### Attachment and Culture of RPE and Other Neuronal Cells onto the Coated Microcarriers

RPE cells are grown in a 60 mm tissue culture dish previously coated with extracellular matrix (ECM) derived from bovine corneal endothelial cells. The RPE cells are fed every other day with culture media containing 15% fetal calf serum (FCS) and bFGF at a concentration of 100 ng/ml. At confluency, the media is changed and 5 ml of fresh medium is added. Then 5−10×10⁶ microcarrier beads which are previously coated with DLC or other combinations are added to the dish. The dish is swirled 8-10 times in a figure-8 motion to endure most of the beads are well distributed, and is then incubated at 37° C. in 10% CO₂ and the microcarriers are allowed to settle at the bottom in direct contact with the RPE cells. A solution of bFGF at concentrations of 100 ng/ml is added every other day to the culture, and 2.5 ml of media will be aspirated very carefully from the top with great care to disturb the microcarriers as little as possible. The layer of RPE cells from the dish will gradually attach to the microcarrier beads and start to proliferate around it until it forms a layer covering the total surface area of the microcarrier beads in 7 to 10 days after the beads are introduced to the culture dish. The microcarriers are then gently detached from the cell layer and further cultured in a roller bottle for 3 days, after which, they are ready to be used for injection into the brain stem for the cell transplantation procedure.

Having described the invention, many modifications thereto will become apparent to those skilled in the art to which it pertains without deviation from the spirit of the invention as defined by the scope of the appended claims.

The disclosures of U.S. Patents, patent applications, and all other references cited above are all hereby incorporated by reference into this specification as if fully set forth in its entirety.

1) A composition useful for the prevention, inhibition or treatment Parkinson's disease in a mammal comprising:
a) live pigmented cells derived from the substantia nigra area of the brain of a mammal or the retinal pigmented epithelium layer or a mammal; and
b) a biodegradable polymer gel capable of photo-induced cross linking.

2) The composition of claim 1 wherein said biodegradable polymer gel further comprises a water soluble macromer having poly(ethylene glycol)di-ethylphosphatidyl(ethylene glycol)methacrylate.

3) The composition of claim 2 wherein said biodegradable polymer gel further comprises attachment proteins and growth factors to enhance the survival of pigmented cells after implantation.

4) The composition of claim 3 wherein said attachment proteins can be laminin, fibronectin, and RGDS.

5) The composition of claim 1 wherein the live pigmented cells are mixed with the polymer gel solution (10 to 20% W/V).

6) The composition of claim 5 wherein the concentration of live pigmented cells is at least 200,000 cells to about 800,000 cells.

7) The composition of claim 3 wherein said growth factors are bFGF and EGF.

8) The composition of claim 7 wherein said growth factors are conjugated to polycarbophyll.

9) The composition of claim 1 wherein said biodegradable polymer gel further comprises a water soluble comprising a Poly-vinyl alcohol.

10) A method for the prevention, inhibition or treatment Parkinson's disease in a mammal comprising:
a) harvesting pigmented cells (Human or bovine origin) from the brain stem (substantia nigra area) or from the retinal pigmented epithelium layer;
b) maintaining said cells on BCE-ECM extracellular matrix coated dishes and suitable growth media;
c) harvesting at least 200,000 of said cells;
d) preparing a mixture comprising a biodegradable polymer gel capable of photo-induced cross linking;
e) mixing the live pigmented cells with the polymer gel solution (10 to 20% W/V);
f) introducing into the brain of a mammal mixture of live pigmented cells with the polymer gel solution; and
g) photo-polymerizing the polymer gel using UV light with a photoinitiator.

11) The method of claim 10, wherein said biodegradable polymer gel further comprises attachment proteins and growth factors to enhance the survival of pigmented cells after implantation.

12) The method of claim 10, wherein said attachment proteins can be laminin, fibronectin, and RGDS, and wherein said growth factors are bFGF and EGF.

13) The method of claim 10, wherein said biodegradable polymer gel further comprises a water soluble comprising a Poly-vinyl alcohol.

14) The method of claim 10, wherein said introduction into the brain of a mammal comprises injecting into the brain of a mammal the mixture of live pigmented cells with the polymer gel solution using a needle means.

15) A composition useful for the prevention, inhibition or treatment a retinal cell disease in a mammal comprising:
a) live pigmented cells derived from the retina of a mammal; and
b) a biodegradable polymer gel capable of photo-induced cross linking.

16) The composition of claim 15 wherein said biodegradable polymer gel further comprises a water soluble macromer having poly(ethylene glycol)di-ethylphosphatidyl(ethylene glycol)methacrylate.

17) The composition of claim 16 wherein said biodegradable polymer gel further comprises attachment proteins and growth factors to enhance the survival of pigmented cells after implantation.

18) The composition of claim 17 wherein said attachment proteins can be laminin, fibronectin, and RGDS.

19) The composition of claim 15 wherein the live pigmented cells are mixed with the polymer gel solution (10 to 20% W/V).

20) The composition of claim 19 wherein the concentration of live pigmented cells is at least 200,000 cells to about 800,000 cells.

21) The composition of claim 17 wherein said growth factors are bFGF and EGF.

22) The composition of claim 21 wherein the growth factors are conjugated to polycarbophyll.

23) The composition of claim 15 wherein said biodegradable polymer gel further comprises a water soluble comprising a Poly(vinyl alcohol).

24) A method for the prevention, inhibition or treatment of a retinal cell disease in a mammal comprising:
a) harvesting pigmented cells (Human or bovine origin) from the retinal pigmented epithelium layer;
b) maintaining said cells on BCE-ECM extracellular matrix coated dishes and suitable growth media;
c) harvesting at least 200,000 of said cells;
d) preparing a mixture comprising a biodegradable polymer gel capable of photo-induced cross linking;
e) mixing the live pigmented cells with the polymer gel solution (10 to 20% W/V);
f) introducing into the retina of a mammal mixture of live pigmented cells with the polymer gel solution; and
g) photo-polymerizing the polymer gel using UV light with a photoinitiator.

25) The method of claim 24, wherein said biodegradable polymer gel further comprises attachment proteins and growth factors to enhance the survival of pigmented cells after implantation.

26) The method of claim 24, wherein said attachment proteins can be laminin, fibronectin, and RGDS, and wherein said growth factors are bFGF and EGF.

27) The method of claim 24, wherein said biodegradable polymer gel further comprises a water soluble comprising a Poly(vinyl alcohol).

28) The method of claim 24, wherein said introduction into the retina of a mammal comprises injecting into the retina of a mammal the mixture of live pigmented cells with the polymer gel solution using a needle means.
